(19) United States
(12) Patent Application Publication
(10) Pub. No.: US 2006/0241346 A1
(43) Pub. Date: Oct. 26, 2006

(54) Mounting method for mounting endless belt to self-propelled colonoscope and mounting tool

(76) Inventor: Masazumi Takada, Chiba (JP)

Correspondence Address: YOUNG & THOMPSON, 745 SOUTH 23RD STREET, 2ND FLOOR, ARLINGTON, VA, 22202, US

(21) Appl. No.: 11/406,258

(22) Filed: Apr. 19, 2006

(30) Foreign Application Priority Data
Apr. 21, 2005 (JP) 2005-123368
May 23, 2005 (JP) 2005-149142
Sep. 22, 2005 (JP) 2005-275610
Dec. 19, 2005 (JP) 2005-364274

(51) Int. Cl. A61B 1/00

(52) U.S. Cl. 600/130; 600/920; 600/128; 600/104

(57) ABSTRACT

A colonoscope (1) is equipped with a plurality of endless belts (17) at circulating passes around an outside and an inside of a flexible section (15) thereof and also equipped with a driving mechanism for driving the endless belts. Each of the endless belt (17) is mounted to the colonoscope (1) such that a separated belt is passed through the circulating pass and then the both ends of the separated endless belt are adhered with an adhesive. At this point, the lead and tail ends of the separated endless belt are placed in a positioning groove formed on a mounting tool and then adhered face to face each other. Thus, the endless belt can be mounted to the flexible section of the colonoscope easily and securely.

### FIELD OF THE INVENTION

The present invention relates to a mounting method for mounting an endless belt onto a self-propelled colonoscope easily, in which the self-propelled colonoscope is equipped with a plurality of endless belts at an outer surface of a flexible section of an insertion tube thereof and inserted into a colon by driving the endless belts, and a mounting tool used in the mounting method.

### BACKGROUND OF THE INVENTION

Colonoscopic examination is currently carried out by inserting a colonoscope into a colon by hand, so the examination may cause pain to a patient by excessively extending or bending the colon. On the other hand, as a colonoscope which does not cause pain to a patient, self-propelled colonoscopes along a colon have been proposed.

Japanese Patent No. 3514252 proposed by the inventor discloses a self-propelled colonoscope which is equipped with a plurality of endless belts at an outer surface of a flexible section of an insertion tube thereof and inserted into a colon by driving the endless belts. The endless belts contact a wall of the colon outside the flexible section and pass through guide pipes, mounted inside the flexible section in the length direction. By driving the endless belts by a driving mechanism, the endless belts are endlessly rotated so as to contact the wall of the colon outside the flexible section and pass through the guide pipe inside the flexible section. As a result, friction between the endless belts and the wall of the colon leads to the tip of the colonoscope into the colon. So, the colonoscope propels into the colon without excessive extension and bending of the colon. Therefore, the colonoscope is smoothly inserted into a colon while remaining the colon relatively intact, whereby a patient hardly senses pain.

Since such a colonoscope contacts body fluid of the patient during the examination, many sections thereof should be cleaned after the examination. Especially, the endless belts directly contact the wall of the colon thus to be contaminated with body fluid and stool. And, the guide pipes through which the endless belts pass and the main body containing driving rollers for driving the endless belts are also contaminated.

Accordingly, the colonoscope is designed such that each endless belt is separable at an intermediate portion thereof and removable from the main body of the colonoscope so as to clean the endless belts and the main body. When new or cleaned endless belts are equipped for the main body, the new or cleaned separated belts are placed to the flexible section of the colonoscope and then both ends of each belt are re-connected.

### SUMMARY OF THE INVENTION

An object of the invention is therefore to provide a method for mounting an endless belt onto a main body of a colonoscope easily and a mounting tool used in the method.

A method for mounting an endless belt onto a self-propelled colonoscope according to the present invention is a method for mounting an endless belt onto a colonoscope, in which the colonoscope is equipped with a plurality of endless belts at circulating passes around an outside and an inside of a flexible section thereof and also equipped with a driving mechanism for driving the endless belts,

wherein a separated endless belt is mounted in such a manner that the belt is passed through said circulating pass from one end (a leading end) thereof and then both ends (the leading end and a tail end) of the belt are adhered face to face by using a mounting tool to form an endless belt, in which the mounting tool is provided with positioning grooves for placing the both end of the belt face to face.

Since the endless belt has a small diameter as small as 1 to 3 mm as described later, it is difficult to butt both ends of the separated endless belt coaxially by hands. So, the mounting tool capable of positioning the both ends of the separated endless belt allows easy and secure butting of the both ends of the separated endless belt.

In the present invention, the positioning groove formed on the mounting tool and their neighboring area are preferably coated with an anti-adhesive, thereby to prevent the separated endless belt from adhering to the mounting tool.

The use of an adhesive is preferably suitable for connecting both ends of the separated endless belt. Consequentially, when the both ends of the separated endless belt are placed in the positioning groove and the end faces thereof are coated with an adhesive, the positioning grooves and their neighboring area are preferably coated with an anti-adhesive so as to prevent the belt from adhering to the mounting tool with the adhesive. Alternatively, the mounting tool may be made of solid Teflon (trade name).

A tool for mounting an endless belt onto a self-propelled colonoscope according to the present invention, in which the colonoscope is equipped with endless belts at circulating passes around an outside and an inside of a flexible section thereof and also equipped with a driving mechanism for driving the endless belts, comprises: a pair of plates each formed with a positioning groove in which both ends of a separated endless belt are placed, and open-close means for pivoting said plates such that said positioning grooves are communicated each other at closing.

The mounting tool according to the present invention is used as follows. Both ends of a separated endless belt are placed on a positioning groove formed on one of the plates with end faces thereof being faced each other. And, the end faces are coated with an adhesive and then butted each other. Then, the other plate is pivoted onto the former plate such that the positioning grooves are communicated each other, resulting in connecting the both ends of the belt coaxially.

In the present invention, the plates are preferably coupled by a hinge, and one of the plates preferably has a thickness capable of being inserted into a clearance between the flexible section of the self-propelled colonoscope and the endless belt mounted on the outer surface of the flexible section.

A work for re-connecting the both ends of the separated endless belt is carried out at an outside of the flexible section after placing the belt around the flexible section in the length direction. In this case, one of the plates is necessarily inserted in a clearance between the belt and the flexible section. Since the endless belt is tightly arranged on the flexible section, the clearance between the endless belt and the flexible section is narrow. So, the plate preferably has a thickness capable of being inserted into the clearance.

As described above, according to the present invention, a method for mounting an endless belt onto a flexible section of a colonoscope easily and firmly, and a tool used in the method are provided.

### BRIEF DESCRIPTION OF THE DRAWINGS

FIG. 1 is a perspective view showing a self-propelled colonoscope (example) according to one embodiment of the present invention.

FIG. 2 is a side sectional view schematically showing the driving unit of the colonoscope according to the invention.

FIG. 3(A) is a perspective view schematically showing a structure of the endless belt of the colonoscope according to the present invention, FIG. 3(B) is a perspective view schematically showing a pulley on which the endless belts are winded, and FIG. 3(C) is a sectional view showing an engagement state between the endless belt and the pulley.

FIG. 4 is a sectional view showing the flexible section of the insertion tube of the colonoscope according to the invention.

FIG. 5 is a front sectional view showing the distal section of the insertion tube of the colonoscope according to the invention.

FIG. 6 is a side view showing a portion around the distal end of the flexible section of the insertion tube of the colonoscope according to the invention.

FIG. 7 is a view showing a belt arrangement jig.

FIG. 8 is a view showing a method for mounting the endless belt by using the belt arrangement jig.

FIG. 9 is a view showing the mounting tool.

FIG. 10 are views showing a method for connecting both ends of the separated endless belt using the mounting tool, FIG. 10A is a plane view and FIG. 10B is a side view.

### DESCRIPTION OF THE PREFERRED EMBODIMENT

Referring to the attached drawings, details of the embodiments of the present invention will be set forth.

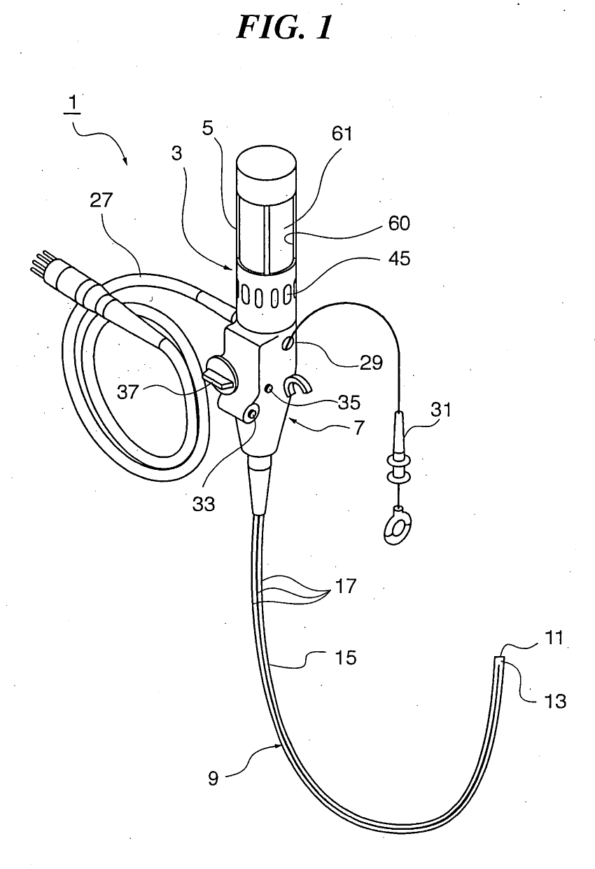

FIG. 1 is a perspective view showing a self-propelled colonoscope (example) according to one embodiment of the present invention.

A self-propelled colonoscope 1 is provided with a belt driving unit 5 covered with a driving unit body 3, an operation unit 7 under the belt driving unit, and an insertion tube 9 extending from the operation unit 7. The insertion tube 9 comprises a distal section 11, a bending section 13, and a flexible section 15. A plurality of endless belts 17 is longitudinally arranged on the flexible section 15.

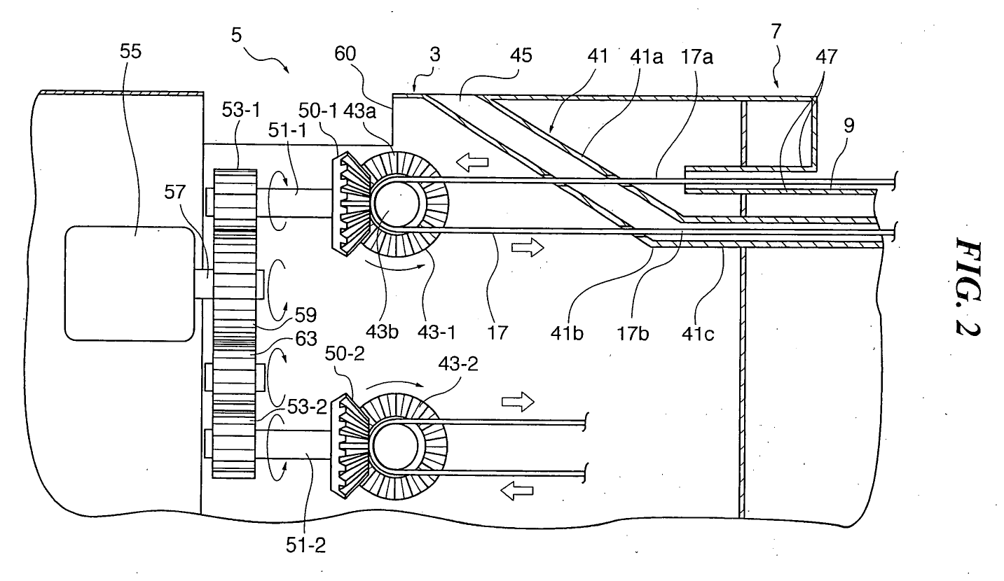

FIG. 2 is a side sectional view schematically showing the driving unit of the colonoscope according to the invention.

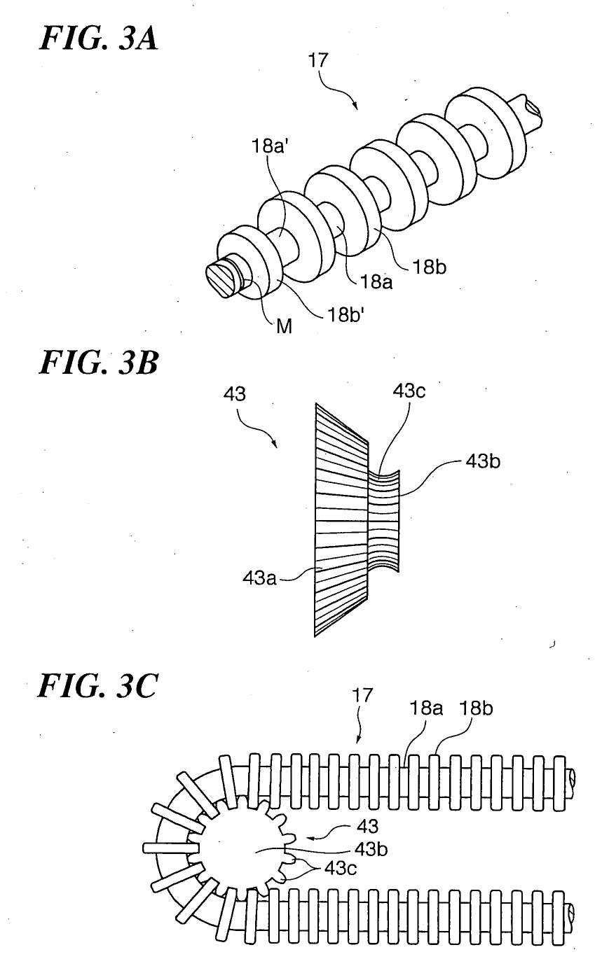

FIG. 3(A) is a perspective view schematically showing a structure of the endless belt of the colonoscope according to the present invention, FIG. 3(B) is a perspective view schematically showing a pulley on which the endless belts are winded, and FIG. 3(C) is a sectional view showing an engagement state between the endless belt and the pulley.

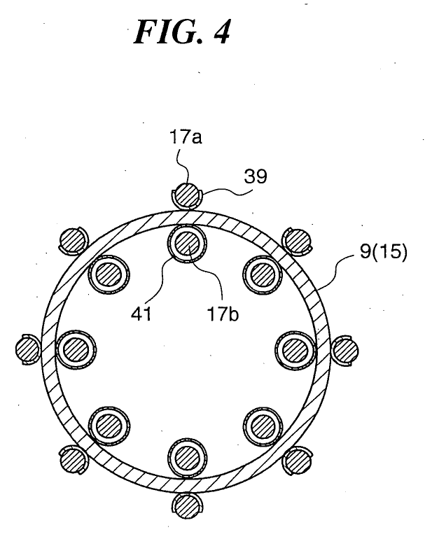

FIG. 4 is a sectional view showing the flexible section of the insertion tube of the colonoscope according to the invention.

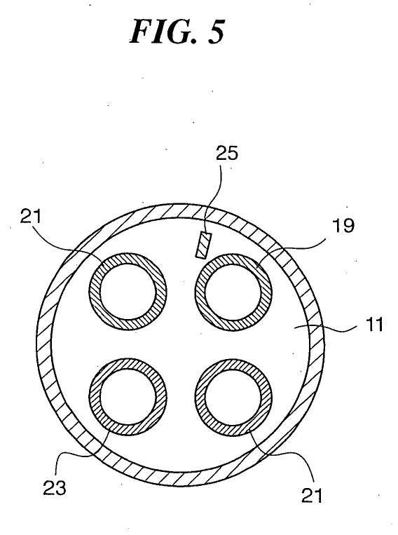

FIG. 5 is a front sectional view showing the distal section of the insertion tube of the colonoscope according to the invention.

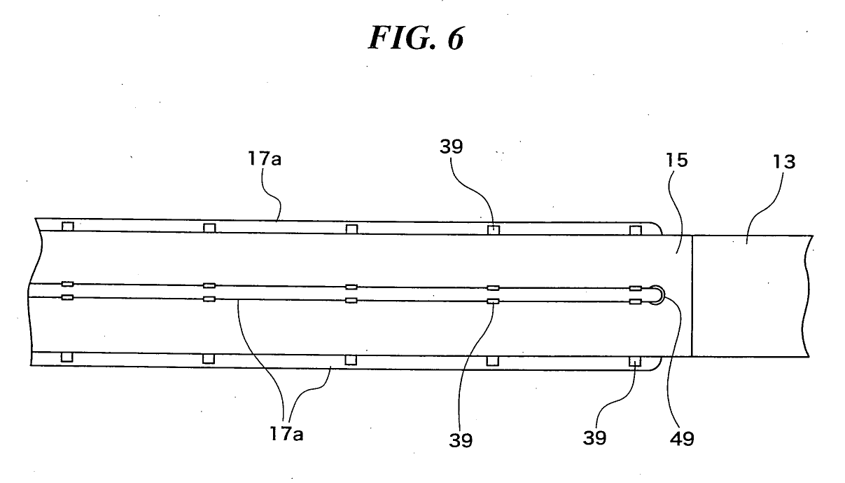

FIG. 6 is a side view showing a portion around the distal end of the flexible section of the insertion tube of the colonoscope according to the invention.

At the distal section 11 of the insertion tube 9, as shown in FIG. 5, an image receiving window 19, one or two light projecting windows 21, a suction and forceps opening 23, and an air-water nozzle 25 are formed. The image-receiving window 19, equipped with an objective lens when the observation device is a fiber scope, or an image pick-up device, such as a CCD, when it is an electronic scope, receives an image from the distal end surface thereof. The received image is transmitted to the operation unit 7 through an image guide of the fiber scope or the lead wire of the electronic scope, which is inserted in the insertion tube 9, and then transmitted through a universal cord 27 to a display and the like to be displayed. A light guide, such as an optical fiber, is inserted in the bore of each of the light projecting windows 21, runs through the operation unit 7 and is connected to an external light source via the universal cord 27. The light source projects light from the distal end surface of the light guide.

The suction and forceps opening 23 is connected to a forceps insertion opening 29 (referring to FIG. 1) formed on the operation unit 7 and a forceps 31 is inserted therein. A working tip end of the forceps 31, protruding from the distal end of the insertion tube 9, is manipulated at the proximal part of the forceps 31 to perform procedures such as to treat a lesion of a patient or to collect a tissue from a patient.

The bore of the air-water nozzle 25 is a water-air supply channel, and air or a cleaning solution is injected through the air-water nozzle 25 by manipulating the air-water supply button 33 mounted at the operation unit 7. Through the suction and forceps opening 23, body fluid or cleaning solution remaining in the colon is sucked out and discharged to outside of the patient's body. This operation is carried out by manipulating a suction control button 35 mounted at the operation unit 7.

The bending section 13 of the insertion tube 9 is able to bend upward and downward, rightward and leftward, and obliquely by manipulating a control knob 37 provided at the operation unit 7.

A plurality of endless belts 17 is longitudinally arranged on the outer surface of the flexible section 15 of the insertion tube 9. The diameter of the flexible section 15 is preferably 5 to 30 mm; 16 mm is most preferable. A larger number of the endless belt 10 is preferable, because the larger the number of the endless belts is, the higher the self-propelling ability of the colonoscope becomes. As shown in FIG. 4, the exterior portion 17a of the endless belt 17 is supported by guide hooks 39 formed on the outer surface of the insertion tube 9. The interior portion 17b of the endless belt 17 passes through a guide pipe 41 mounted on the inner surface of the flexible section 15. The guide hooks 39 and the guide pipe 41 constitute a circulating pass of the endless belt 17. The guide hooks 39, each having a cross section of circular-arc of which inner angle is over 180°, are mounted at the flexible section 15 in the length direction, and the endless belts 17 are radially and outwardly exposed from the guide hooks 39 (as shown in FIG. 6). Accordingly, the exterior portion of the endless belt 17 supported by the guide hook 39 is exposed from the guide hook 39 so as to contact the inner wall of the colon with a sufficient contact area when the flexible section 15 is inserted into the colon. Even when the flexible section 15 is severely bent, the endless belts 17 will not come off the guide hooks 39.

In addition, the guide hooks 39 are formed in the length direction of the flexible section 15 at intervals of 2 to 3 cm. However, the guide hook 39 may be continuously formed in the length direction of the flexible section. The guide hook 39 is made of an elastic material for spreading the opening thereof.

Each of the endless belts 17 is made of a flexible and strong material such as, for instance, carbon fiber or resin, and, as shown in FIG. 3A and FIG. 3C, comprises an axial belt 18a and a plurality of rack gear teeth 18b arranged along the length direction of the axial belt 18a. The axial belt 18a has a cross section of circle having a diameter of 1 to 3 mm. The rack gear teeth 18b also have a cross section of circle and are coaxially arranged at regular intervals on the outer surface of the axial belt 18a. The rack gear teeth 18b has a diameter of 1 to 3 mm and a thickness of 0.1 to 1.0 mm. And, a distance between any two teeth of the rack gear teeth 18b is 0.1 to 11.0 mm. The outer surface of the rack gear teeth 18b may be coated with high frictional material. And, the outer surface of the pulley 43c including a pinion gear teeth 43c, as described later, may be coated with high frictional material. The length of the endless belt 17 will be described later.

The endless belt 17 is formed by adhering both ends of a single belt. The endless belt is removed from the insertion tube 9 for cleaning the colonoscope. When the endless belt is removed, the endless belt 17 is separated at an intermediate portion thereof. The intermediate portion, at which the endless belt 17 is separated, is marked with a mark M at a circumferential surface thereof. And, one of the rack gear teeth 18b′ next to the mark has a diameter smaller than the other rack gear tooth. A method for removing the endless belt and re-mounting thereof will be explained later.

The endless belt has a circular cross section so as to be able to be flexibly bent in all radial directions to the axis thereof with equal forces. So, when the insertion tube is inserted along the bending of the colon, the endless belt can be easily followed the motion of the insertion tube. Since the rack gear teeth are formed along the entire length of the endless belt, in case of twisting of the endless belt, the rack gear teeth may contact the wall of the colon at any portion thereof so that the endless belt can be rubbed against the wall of the colon. Accordingly, the friction force between the endless belts and the wall of the colon will be increased thereby to improve the self-propelling ability.

Next, referring to FIG. 2, constructions of the proximal part of the guide pipe 41 and the belt driving unit 5 will be explained.

The proximal part of the guide pipe 41 is connected to a guide pipe opening 45 formed on the side surface of the driving unit body 3. The driving unit body 3 is larger than the insertion tube 9 in diameter. The guide pipe 41 comprises an inclined section 41a, extending diagonally from the guide pipe opening 45 to the insertion tube 9, and a guide section 41c, extending straight in the insertion tube 9 from the inclined section 41a through a bending section 41b.

The drive roller 43 to which the endless belt 17 is held is mounted at a proximal side of the guide pipe 41 in the driving unit body 3. The endless belt 17 penetrates the sidewalls of the guide pipe 41 at the inclined section 41a of the guide pipe 41. That is, the exterior portion 17a of the endless belt 17 runs through a guide section 47 from the outer surface of the insertion tube 9 and is entered into the driving body 3 through holes formed at the two side walls of the inclined section 41a of the guide pipe 41. Then, the endless belt 17 is winded around the drive roller 43. And then, the interior portion 17b of the endless belt 17 runs through the guide pipe 41 from a hole formed at the side wall of the guide pipe 41 at the inclined section 41a toward a guide hole 49 (as shown in FIG. 6) formed near the distal end of the flexible section 15 of the insertion tube 9.

The guide hole 49, as shown in FIG. 6, is preferably positioned at 0 to 10 cm from the distal end of the flexible section 15. This is because the larger the surface where the inside wall of the colon is in contact with the exterior portion 17a of the endless belt 17 is, the higher the self-propelling ability of the self-propelled colonoscope becomes.

On the other hand, the distal section of the insertion tube 9, inserted into the colon, advances from the sigmoid colon to the ileum through the descending colon, the transverse colon and the ascending colon in the above described manner. Since the flexible section has an outer diameter as lager as about 16 mm, when the distal end of the colonoscope advances into the colon, the curve of the colon causes a difference in length of the inserted flexible section 15 between the inner ring length and the outer ring length thereof. When the distal end of the insertion tube reaches the ileum and thus the flexible section 15 having a diameter of 16 mm turns in a circular pattern, the outer ring length is 3.12% longer than the straight length.

Accordingly, the endless belt 17 arranged on the surface of the flexible section 15 necessarily has an allowable length for difference between the inner ring length and the outer ring length of the flexible section 15. For this reason, the length of the endless belt 17 is designed to have a length of 102 to 104% of the length of such belt when it tensely circulates from the guide hole 49 near the tip of the flexible section 15 to the same guide hole 49 through the driving unit, while the flexible section 15 is kept straight. Since the length of the endless belt 17 is set as above, the endless belt 17 can sufficiently follow the bending of the flexible section 15. Therefore, the colonoscope can advance into the colon stably.

The drive roller 43 is provided with a pulley 43b on which the endless belt 17 is winded and a bevel gear 43a connected to the same shaft as the pulley 43b. On the circumferential side surface of the pulley 43b, as shown in FIG. 3C, a concave groove is formed. And, in the concave groove, the pinion gear teeth 43c are formed, which are engaged with the rack gear teeth 18b of the endless belt 17.

As mentioned above, the endless belt 17 has a length to have a certain of allowance. However, since the pinion gear teeth 43c are formed on the circumferential side surface of the pulley 43b for driving the endless belt 17, the endless belt 17 and the pulley 43b are tightly engaged each other by engagement of the rack gear teeth 18b and the pinion gear teeth 43c, whereby the endless belt can be driven without idling.

Referring to FIG. 2 again, a bevel gear 50 is arranged orthogonal to the bevel gear 43a and engaged therewith. A spur gear 53 is fixedly mounted at the proximal end of a gear shaft 51 of the bevel gear 50. The spur gear 53 is engaged with a large spur gear 59 fixedly mounted to a motor shaft 57 of a motor 55. Consequently, when the motor shaft 57 revolves by driving the motor 55, the bevel gear 43a will revolve, via the large spur gear 59, the spur gear 53, and the bevel gear 50, and therefore the pulley 43b will revolve together.

On the circumferential side surface of the large spur gear 59, the same number of drive rollers 43, bevel gears 50, gear shafts 51, and spur gears 53, as endless belts 17, are mounted. Incidentally, an intermediate gear 63 may be provided between the large spur gear 59 and the spur gear 53 in order to drive each of the endless belts 17 in the same direction.

The motor 55, the large spur gear 59, the spur gear 53, the gear shaft 51, the bevel gear 50, and the drive roller 43 are housed in the driving unit body 3 on the proximal side of the guide pipe opening 45. On the side surface of the driving unit body 3, a cleaning opening 60 is formed. The opening 60 is provided with a lid 61 for opening and closing the opening 60, as shown in FIG. 1. The opening 60 is opened to a space containing the large spur gear 59, the spur gear 53, the gear shaft 51, the bevel gear 50, and the drive roller 43. The space containing the large spur gear 59, the spur gear 53, the gear shaft 51, the bevel gear 50, and the drive roller 43, and a space containing the motor 55 are water-tightly isolated.

When the motor 55 is driven to rotate the pulley 43b counterclockwise, the exterior portion 17a of the endless belt 17 engaged with the pulley 43b will rotate to the left direction in the figure. In a case which the exterior portion 17a of the endless belt 17 is in contact with the inside wall of the colon, the insertion tube 9 will advance forward to the right direction in FIG. 2 by friction between the endless belts 17 and the inside wall of the colon. The insertion tube 9 moves back by rotating the motor 55 clockwise.

When the colonoscope is cleaned, the endless belt 17 is separated at an intermediate portion thereof. The endless belt 17 is separated at the marked portion of the axis belt 18a. Then, the separated endless belt is removed from the colonoscope. And, after cleaning the separated endless belts and the main body of the colonoscope, the separated endless belts are re-mounted on the colonoscope. Hereinafter, a method for mounting the separated endless belt on the colonoscope will be explained.

First, the separated endless belt is arranged on the flexible section of the colonoscope by using a belt arrangement jig.

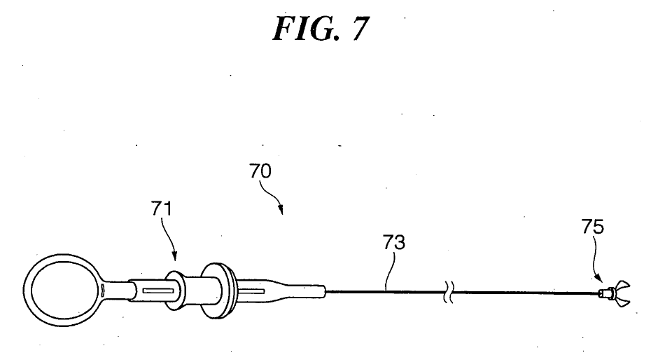

FIG. 7 is a view showing a belt arrangement jig.

The belt arrangement jig 70 is formed of a forceps providing with an operating section 71, an insertion section 73 and a working section 75. And, the working section 75 is operatable by the operating section 71. The insertion section 73 is made of a flexible material and has a length longer than the endless belt 17.

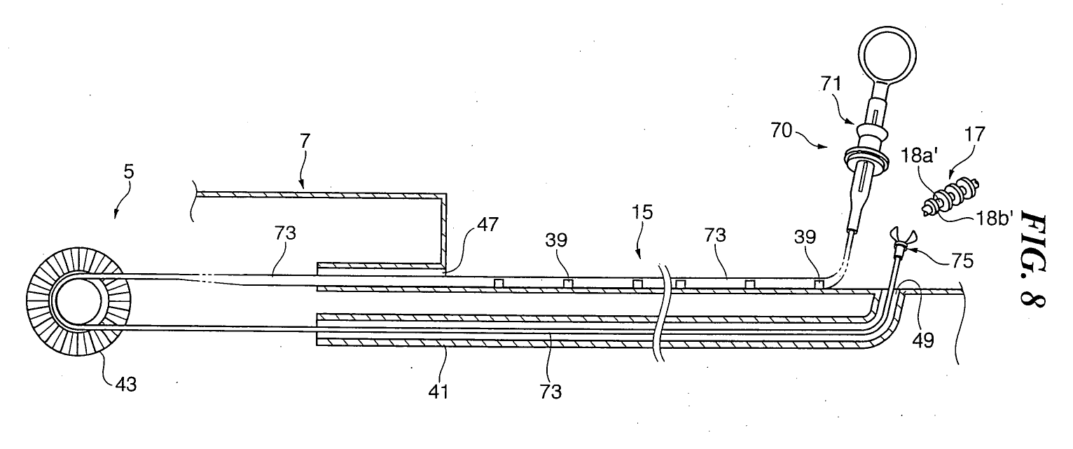

FIG. 8 is a view showing a method for mounting the endless belt by using the belt arrangement jig.

First, the jig 70 of the working section 75 with being closed is run through the guide hook 39 formed on the outer surface of the flexible section 15 of the colonoscope from the distal end of the flexible section 15 (the right side in the figure) toward the operating portion 7 (the left side in the figure). When the working section 75 reaches the operating portion 7, the working section 75 is advanced into the belt driving unit 5 through the operating portion 7 from the guide section 47 and then is turned around the driving roller 43 therein. Then, the working section 75 is advanced through the guide pipe 41, mounted on the inner surface of the flexible section 15, to the distal end of the flexible section 15 and come out through the guide hole 49 from the guide pipe. That is, the insertion section 73 of the jig 70 goes around the circulating pass of the endless belt through the outside and the inside of the flexible section 15.

Then, the operating section 71 is operated such that the working section 75, come out through the guide hole 49, gripes the axis belt 18a′ next to the small diameter rack gear tooth 18b at one end of the separated endless belt 17. Since the rack gear tooth 18b next to the axis belt 18a′ which is gripped by the working section 75 is designed to have a small diameter, the working section 75, which gripes the axis 18a′, has a small diameter almost the same as the diameter of the endless belt 17. And, the insertion section 73 of the jig (the forceps) is removed out of the flexible section 15 of the colonoscope through the outside and the inside of the flexible section 15 with the working section 75 gripping the axis 18a′. In this case, since the gripping section 75 which grips the axis 18a′ has almost the same diameter as the endless belt 17, the jig 70 can be removed out smoothly without bothering to enlarge the diameter of the guide pipe 41. As a result, the tip of the separated endless belt 17 will pass into the guide pipe 41 from the guide hole 49 formed on the distal end of the flexible section 15 and turn around the driving roller 43 in the belt driving unit 5. Then, the tip end of the separated endless belt 17 passes through the guide hooks 39 formed on the outer surface of the flexible section 15 from the guide section 47 of the operating portion 7 to the distal end of the flexible section 15. That is, the separated endless belt is pulled by the jig 70 so as to make a circuit through the inside and the outside of the flexible section 15.

Then, both ends of the separated endless belt 17 thus arranged around the flexible section 15 are connected each other using a mounting tool.

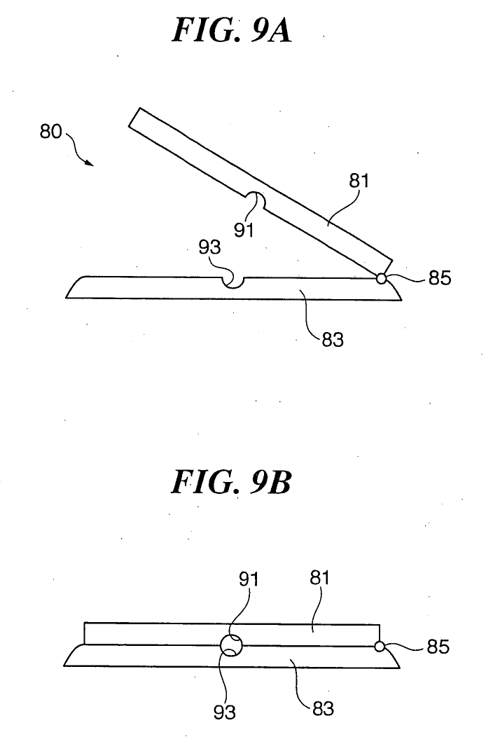

FIG. 9 is a view showing the mounting tool.

The mounting tool 80 is provided with an upper positioning plate 81 and a lower positioning plate 83 which are coupled openably and closably by a hinge 85. Both positioning plates 81 and 83 are formed with positioning groove 91 and 93 on which the separated endless belt is placed. When the upper positioning plate 81 is closed toward the lower positioning plate 83, the upper positioning groove 91 is communicated with the lower positioning groove 93. Each of the positioning grooves 91 and 93 has a semicircular, section. So, when the upper positioning plate 81 is closed toward the lower positioning plate 83, both positioning grooves form a circular section having a diameter slightly larger than a diameter of the rack gear teeth 18b of the separated endless belt 17.

The upper and lower plates 81 and 83 are preferably made of a strong anti-adhesive material such as solid Teflon (Trade Name) and the like. Alternatively, the positioning grooves 91 and 93 and their neighboring area are preferably coated with a strong anti-adhesive. When an instant adhesive, for example, Aronalpha A, (Trade Name) manufactured by Sankyo Co., Ltd., usable for medical treatment is used for connecting of the separated endless belt 17, the anti-adhesive may include acetone, dimethyl formamide, dimethyl sulfoxide, silicon resin and fluorocarbon resin.

And, the lower positioning plate 83 has a thickness (for example 10 mm) capable of being inserted into a clearance between the flexible section 15 of the colonoscope and the endless belt 17 arranged on the outer surface of the flexible section. Since the endless belt 17 has a length of 102 to 104% of the length of such belt when it tensely turns around from the guide hole 49 near the tip of the flexible section 15 to the same guide hole 49 through the driving unit, while the flexible section 15 is kept straight, a narrow clearance exists between the endless belt 17 and the flexible section 15. Accordingly, the lower positioning plate necessarily has a thickness capable of being inserted into the narrow clearance.

Next, a method for connecting both ends of a separated belt using the mounting tool will be explained.

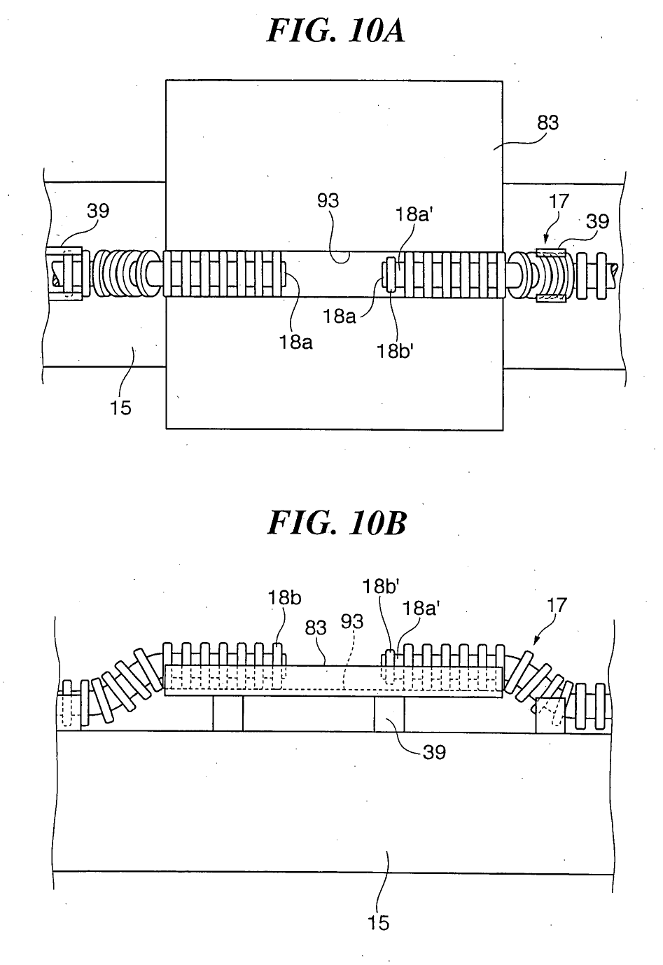

FIG. 10 are views showing a method for connecting both ends of the separated endless belt using the mounting tool, FIG. 10A is a plane view and FIG. 10B is a side view.

First, the lower positioning plate 83 of the mounting tool 80 is placed on the outer surface of the flexible section 15, and both ends of the separated endless belt 17 are placed on the positioning groove 93 of the lower positioning plate 83 with end surfaces thereof being faced. And, both the end surfaces 18a are coated with adhesive and butted each other. At the same time, the upper positioning plate 81 (not shown) is closed toward the lower positioning plate 83. As a result, both end surfaces 18a of the separated endless belt 17 are coaxially connected with the adhesive.

Since the positioning grooves 91 and 93 and their neighboring area are coated with anti-adhesive, the adhesive protruding from the connected surface (end surface) does not adhere to the both plates.

After curing the adhesive and connecting the both end surfaces 18a with the adhesive, the lower positioning plate 83 is removed from the clearance between the endless belt 17 and the flexible section 15 and, the mounting tool 80 is retrieved.

What is claimed is:

1. A method for mounting an endless belt onto a self-propelled colonoscope, in which the colonoscope is equipped with a plurality of endless belts at circulating passes around an outside and an inside of a flexible section thereof and also equipped with a driving mechanism for driving the endless belts,
wherein a separated endless belt is mounted in such a manner that the belt is passed through said circulating pass from one end (a leading end) thereof and then both ends (the leading end and a tail end) of the belt are adhered face to face by using a mounting tool to form an endless belt, in which the mounting tool is provided with a positioning groove for placing the both end of the belt face to face.

2. A method for mounting an endless belt onto a self-propelled colonoscope according to claim 1,
wherein said positioning grooves formed on the mounting tool and their neighboring area are coated with an anti-adhesive, thereby to prevent the separated endless belt from adhering to said mounting tool.

3. A tool for mounting an endless belt onto a self-propelled colonoscope, in which the colonoscope is equipped with endless belts at circulating passes around an outside and an inside of a flexible section thereof and also equipped with a driving mechanism for driving the endless belts, comprising:
a pair of plates each formed with a positioning groove in which both ends of a separated endless belt are placed, and
open-close means for pivoting said plates such that said positioning grooves are communicated each other at closing.

4. A tool for mounting an endless belt onto a self-propelled colonoscope according to claim 3,
wherein said plates are coupled by a hinge, and
one of the plates preferably has a thickness capable of being inserted into a clearance between the flexible section of the self-propelled colonoscope and the endless belt mounted on the outer surface of the flexible section.
